(19) United States
(12) Patent Application Publication
(10) Pub. No.: US 2005/0238610 A1
(43) Pub. Date: Oct. 27, 2005

(54) Low-viscosity emulsions

(75) Inventors: Jens Nielsen, Henstedt-Ulzburg (DE); Thomas Raschke, Pinneberg (DE); Nadine Voigt, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/040,813

(22) Filed: Jan. 21, 2005

(30) Foreign Application Priority Data
Jan. 21, 2004 (DE) 10 2004 003436.2

(51) Int. Cl. A61K 7/075; A61K 7/08

(52) U.S. Cl. 424/70.31

(57) ABSTRACT

Cosmetic and/or dermatological oil-in-water (O/W) emulsion with a viscosity of 100 to 1900 mPaS, containing a) one or more electrolytes in a concentration of 0.5 to 8% by weight, b) a thickener in a concentration of 0.05 to 1% by weight, and c) an emulsifier combination in a concentration of 0.5 to 6% by weight, which comprises polyethylene glycol stearate esters and at least one further emulsifier selected from polyoxyethylene cetylstearyl ethers and glyceryl stearate, wherein the weight percentages are based on the total weight of the emulsion.

The present invention relates to a low-viscosity oil-in-water (O/W) emulsion.

The desire to look beautiful and attractive is rooted in humans by nature. Even when the ideal of beauty has undergone changes in the course of time, the endeavor for a flawless exterior has always been a goal of humans. In this connection, the condition and the appearance of the skin and the skin appendages form a substantial part of a beautiful and attractive exterior.

In order that the skin and the skin appendages, these include primarily hair and nails, are able to fulfill their biological functions to the full extent, they need regular cleansing and care as well as protection against UV radiation. In this connection, cleansing serves the purposes of removing dirt, sweat, and remnants of deceased body cells, which form an ideal culture medium for all kinds of infectious germs and parasites. Cosmetic cleansing products are normally offered in the form of gels, lotions, and solids (soap bars, syndet soaps). Skin care products, normally creams, ointments, or lotions are used in most cases to moisturize and refat the skin as well as to supply the skin with cosmetic or dermatological agents (for example, vitamins, antioxidants, UV light protection filters). This will be important just when the natural regeneration capacity of the skin does not suffice to make up for stresses and damage to the skin which result from environmental influences, mechanical or chemical irritations, sun light, and wind (and which accelerate, among other things, aging of the skin).

A particularly common form of cosmetic emulsions are low-viscosity oil-in-water emulsions (O/W emulsions). Low-viscosity O/W emulsions are normally based on an emulsifier system of ceteareth-20, ceteareth 12, cetearyl alcohol, cetyl palmitate, and glyceryl stearate. Further O/W emulsifier systems are: triceteareth-4 phosphate; trilaureth-4 phosphate; glyceryl isostearate+isoceteth-20; PEG-60 hydrogenated castor oil, or polysorbate 20.

However, in the art, preparations of this type have the disadvantage that last but not least because of their relatively high emulsifier content, they leave a sticky sensation on the skin and offer little care. Furthermore, such preparations normally have no particularly long shelf life (long-term stability).

Furthermore, highly viscous O/W emulsions with polyethylene glycol esters as emulsifiers are known in the art. Emulsions with polyethylene glycol esters as emulsifiers have the advantage that they are long-term stable and electrolyte-compatible. Furthermore, they leave a rich, nonsticky sensation on the skin, and are well tolerated by the skin.

However, preparations of this type have the disadvantage that they cover only a limited application spectrum because of their viscosity. They are neither sprayable nor suitable as impregnation medium for wipes.

To solve the problems of the art, different approaches have been taken in the past. For example, Pickering pigments were used as emulsifiers (DE 102 38 649.8). A further approach consisted in using a high concentration of emulsifiers in combination with fatty alcohols (DE 198 02 204.6 and DE 101 41 258, as well as EP 1 074 242, EP 1 077 062, and EP 835 651) or W/O emulsions (DE 101 54 627). Fatty alcohols, however, have the disadvantage that they greatly increase the viscosity of O/W emulsions (even though they improve the stability of O/W emulsions), and very greatly influence the sensation on the skin. An increase in stability of the emulsion is thus obtained at the expense of sensory flexibility.

Furthermore, O/W emulsions of the art have the disadvantage that they show only little temperature stability at extreme temperatures and in a greater temperature interval (−10° C. to 40° C.). In particular UV light protective preparations must however withdstand high thermal stresses both in summer (for example, on the beach) and in winter (for example, in skiing).

Furthermore, particularly low-viscosity O/W emulsions of the art permit incorporating only relatively small quantities of electrolytes. Especially electrolyte contents of more than 1% by weight have a tendency to phase separation (in particular under thermal stress).

It was therefore an object of the present invention to develop low-viscosity O/W emulsions, which eliminate the deficiencies of the art.

It was a further object of the present invention to develop low-viscosity, particularly storage-stable O/W emulsions, which leave at most a minimally sticky sensation on the skin, and which are in particular well tolerated by the skin. Furthermore, it was an object of the present invention to develop a particularly temperature-stable, low-viscosity O/W emulsion, which is resistant to temperature and storage over a longer period even in the case of high electrolyte contents.

Last but not least, the O/W emulsion had to meet the requirement of being both suitable as an impregnating preparation for wipes and other textiles, and sprayable by means of a sprayer (for example, aerosol can, pump spray).

Surprisingly, these objects are accomplished by a cosmetic and/or dermatological oil-in-water (O/W) emulsion with a viscosity of 100 to 1900 mPas, containing
a) one or more electrolytes in a concentration of 0.5 to 8% by weight,
b) a thickener in a concentration of 0.05 to 1% by weight, and
c) an emulsifier combination in a concentration of 0.5 to 6% by weight, which comprises polyethylene glycol stearate esters and at least one additional emulsifier selected from polyoxyethylene cetylstearyl ethers and glyceryl stearate, with the weight percentages relating to the total weight of the emulsion.

The O/W emulsions of the invention are surprisingly stable, low-viscosity emulsions, which contain large quantities of electrolytes and leave an excellent sensation on the skin. The emulsions of the invention are stable over three months at room temperature or 40° C.

The viscosity values of the preparations and individual substances as specified within the scope of the present Application, were determined with the aid of a viscosity meter of the type Viskotester VT 02 by Haake (temperature: 20° C., 24 mm spindle diameter, 62 rpm rotor speed).

In accordance with the invention, it is preferred to use as emulsifier c) an emulsifier combination of PEG-100 stearate and ceteareth-20.

In accordance with the invention, it is furthermore preferred to use as emulsifier c) an emulsifier combination of PEG-40 stearate and glyceryl stearate.

Last but not least, it is preferred in accordance with the invention to use as emulsifier c) a combination of PEG-100 stearate, polyoxyethylene(20) cetylstearyl ether, and glyceryl stearate.

If polyethylene glycol stearate esters and polyoxyexthylene cetylstearyl ethers are used as emulsifier combination, it will be advantageous in accordance with the invention, when the weight ratio of the polyethylene glycol stearate esters to the polyoxyethylene cetylstearyl ethers is from 1:10 to 5:1.

If polyethylene glycol stearate esters and glyceryl stearate are used as emulsifier combination, it will be advantageous in accordance with the invention, when the weight ratio of polyethylene glycol stearate esters to glyceryl stearate is from 1:10 to 3:1.

PEG-100 stearate (=polyethyleneglycol(100) monostearate, CAS 9004-99-3) is available, inter alia, from ICI under the trade name MYRJ 59 (P). In accordance with the invention, it is advantageous to use PEG-100 stearate in a concentration from 0.5 to 5% by weight, more preferably in a concentration from 0.5 to 2% by weight, each based on the total weight of the emulsion.

Polyoxythylene(20) cetylstearyl ether (INCI: Ceteareth-20) is available, inter alia, from Henkel under the trade name Emulgin B2. In accordance with the invention, it is advantageous to use polyoxythylene(20) cetylstearyl ether in a concentration from 0.5 to 5% by weight, more preferably in a concentration from 1 to 3% by weight, each based on the total weight of the emulsion.

PEG-40 stearate (actually, polyethylene, glycol(2000) monostearate is available, inter alia, from Goldschmidt AG under the trade name MYRJ 52 P. In accordance with the invention, it is advantageous to use PEG-40 stearate in a concentration from 0.25 to 5% by weight, and more preferably in a concentration from 0.5% to 2.5% by weight, each based on the total weight of the emulsion.

Glyceryl stearate (CAS 123-94-4) is available, inter alia, from Goldschmidt under the trade name Hostaphat KW 340 D. In accordance with the invention, it is advantageous to use glyceryl stearate in a concentration from 0.5 to 5% by weight, and more preferably in a concentration from 1 to 3% by weight, each based on the total weight of the emulsion.

Moreover, the emulsion of the invention may contain further emulsifiers, which may be selected from the list of emulsifiers known from cosmetics.

Preferred embodiments of the present invention contain as thickener one or more compounds selected from the group of carrageen, xanthan gum, polyacrylate, hydroxypropylmethyl cellulose, cellulose ether, acryloyldimethyl taurate/VP copolymer, hydroxyproyl distarch phosphate (CAS No. 113894-92-1).

The carrageen of the invention (INCI Chondrus crispus (Carrageenan), CAS 9000-07-1, is available, inter alia, from FMC under the trade name Gelcarin. In accordance with the invention, it is advantageous to use carrageen in a concentration from 0.1 to 2% by weight, and more preferably in a concentration from 0.2 to 1% by weight, each based on the total weight of the emulsion.

The xanthan gum of the invention (INCI xanthan gum, CAS 11138-66-2) is available, inter alia, from Kelco under the trade name Keltrol F. In accordance with the invention, it is advantageous to use xantham gum in a concentration from 0.01 to 2% by weight, and more preferably in a concentration from 0.05 to 0.5% by weight, each based on the total weight of the emulsion.

In accordance with the invention, it is possible and advantageous to use homopolymers or copolymers of the acrylic acid and/or its derivatives of the following structure: 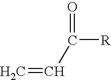
where R6=OH, OR′, NHR′, with R′=alkyl-, aryl-, heteroalkyl-, heteroaryl-, alkyl sulfonates, for example, of the following structure:

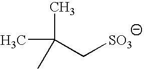

In accordance with the invention, it is possible to use as advantageous comonomers of the advantageous copolymers of the invention, for example: C₁₀-C₃₀-alkyl acrylate, methacrylic acid, and acrylamide.

In accordance with the invention, it is preferred to use the homopolymers of the acrylic acid. More preferably, these have a molecular weight of 2,000,000 to 6,000,000, such as, for example, the commercially available products Permulen® types TR 1, TR 2, and TR Z, Pas 980, 981, 984, 1342, 1382, 2984, and 5984, likewise the EDT types 2001, 2020, 2050, and Carbopol Ultrez 10 from the manufacturer Noveon, as well as Stabileze 06 from ISP.

Hydroxypropylmethyl cellulose (CAS 9004-65-3) is available, inter alia, from Dow Chemical under the trade name Methocel 4EM. It is advantageously used in accordance with the invention in a concentration from 0.05 to 2% by weight, and more preferably in a concentration from 0.2 to 1% by weight, each based on the total weight of the emulsion.

A particularly preferred embodiment of the invention are emulsions, which contain as emulsifier glyceryl stearate citrate in a concentration from 0.5 to 3% by weight, and as thickener a combination of carrageen and xanthan gum at a ratio of 1:5 to 5:1.

According to the invention, electrolytes mean compounds, which are present in the form of water-soluble salts. The cations and anions of the electrolytes may represent both inorganic and organochemical ions. In particular, it is possible to form the salts from anions of inorganic mineral acids (for example, hydrochloric acid, nitric acid, sulfuric acid, carbonic acid, phosphoric acid), and/or organic carbonic acids (also lactic acid, citric acid), and sulfonic acids. As cations one uses in most cases, even though not exclusively, anionic cations, for example, sodium-, potassium-, calcium-, and magnesium ions.

As preferred salts in accordance with the invention, one chooses sodium chloride, magnesium chloride, potassium chloride, taurine, citric acid, lactic acid, ascorbic acid, creatine, salycilic acid, carnitine, as well as UV filter substances from the group of phenylbenzimidazole sulfonic acid, phenylene-1,4-bis-(2-benzimidazyl)-3,3′-5,5′-tetrasulfonic acid, and/or 1,4-di(2-oxo-10-sulfo-3-bornylidenemethyl)-benzene, and/or their salts, in particular phenylene-1,4-bis-(2-benzimidazyl)-3,3′-5,5′tetrasulfonic acid disodium salt, terephthalylidene dicamphor sulfonic acid or its salts, salts of the 2-phenylbenzimidazole-5-sulfonic acid, such as its sodium-, potassium-, or its triethanol ammonium salt, as well as the sulfonic acid itself; and/or sulfonic acid derivatives of the 3-benzylidene camphor, such as, for example, 4-(2-oxo-bornylidenemethyl)benzene sulfonic acid, 2-methyl-5-(2-oxo-3-bornylidenemethyl)sulfonic acid and their salts.

In accordance with the invention, the emulsion of the invention advantageously contains cosmetic and/or dermatological ingredients. In particular, it is preferred in accordance with the invention, that the emulsion contains UV light protective filters and/or cosmetic ingredients.

According to the invention, the emulsions of the invention may advantageously contain as an aqueous phase besides water also other ingredients, for example, alcohols, diols, or polyols of a low carbon number, as well as ethers thereof, preferably ethanol, isopropanol, propylene glycol, glycerol, ethylene glycol, ethylene glycol monoethyl- or -monobutyl ether, propylene glycol monomethyl, -monoethyl-, or -monobutyl ether, diethylene glycol monomethyl or -monoethyl ether, and analogous products, furthermore alcohols of a low carbon number, for example, ethanol, isopropanol, methylpropanediol, 1,2-propanediol, and glycerol.

The emulsion of the invention contains one or more oil, lipid, and/or wax components of identical or different polarity. Polar oils are, for example, those from the group of the lecithins and fatty acid triglycerides, namely the triglycerol esters of saturated and/or unsaturated, branched and/or unbranched alkane carboxylic acids having a chain length from 8 to 24, in particular 12 to 18 carbon atoms. The fatty acid triglycerides may advantageously be selected, for example, from the group of the synthetic, semisynthetic, and natural oils, for example, olive oil, sunflower seed oil, soy oil, peanut oil, rape seed oil, almond oil, palm oil, coconut oil, castor oil, wheat germ oil, grape seed oil, thistle oil, evening primrose oil, macadamia oil, and more of the like.

Particularly advantageous polar lipids for the purposes of the present invention are all native lipids, such as, for example, olive oil, sunflower seed oil, soy oil, peanut oil, rape seed oil, almond oil, palm oil, coconut oil, castor oil, wheat germ oil, grape seed oil, thistle oil, evening primrose oil, macadamia oil, corn oil, avocado oil, and the like, as well as those listed in the following.

| | | | Polarity |
|---|---|---|---|
| Manufacturer | Trade Name | INCI-Name | [mN/m] |
| Condea Chemie | Isofol 14 T | Butyl decanol (+) hexyl octanol (+) | 19.8 |
| | | hexyl Decanol (+) butyl octanol | |
| Lipochemicals | Lipovol MOS-130 | Tridecyl stearate (+) tridecyl | 19.4 |
| INC./USA | | trimellitate(+) dipentaerythrityl | |
| (Induchem) | | hexacaprylate/hexacaprate | |
| | Castor oil | | 19.2 |
| CONDEA Chemie | Isofol Ester 0604 | | 19.1 |
| Huels | Miglyol 840 | Propylene glycol | 18.7 |
| CONDEA Chemie | | dicaprylate/dicaprate | |
| CONDEA | Isofol 12 | Butyl octanol | 17.4 |
| Chemie | | | |
| Goldschmidt | Tegosoft SH | Stearyl heptanoate | 17.8 |
| | Avocado oil | | 14.5 |
| Henkel Cognis | Cetiol B | Dibutyl adipate | 14.3 |
| ALZO (ROVI) | Dermol 488 | PEG 2 diethylenhexanoate | 10.1 |
| Condea Augusta | Cosmacol ELI | C12-13 Alkyl lactate | 8.8 |
| S.P.A. | | | |
| ALZO (ROVI) | Dermol 489 | Diethylene glycol dioctanoate/ | 8.6 |
| | | diisononanoate | |
| Condea Augusta | Cosmacol ETI | Di-C12/13 alkyl tartrate | 7.1 |
| S.P.A. | | | |
| Henkel Cognis | Emerest 2384 | Propylene glycol monoisostearate | 6.2 |
| Henkel Cognis | Myritol 331 | Cocoglycerides | 5.1 |
| Unichema | Prisorine 2041 | Triisostearin | 2.4 |
| | GTIS | | |

Furthermore, the oil phase may advantageously be selected from the group of the dialkyl ethers. It will be particularly advantageous, when the oil phase has a content of C₁₂₋₁₅ alkyl benzoate or totally consists of same.

Furthermore, the oil phase may advantageously be selected from the group of Guerbet alcohols. Guerbet alcohols are named after Marcel Guerbet, who described their production for the first time. They form according to the reaction equation 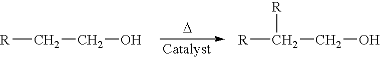
by oxidizing an alcohol to an aldehyde, by aldol condensation of the aldehyde, separation of water from the aldol, and hydrogenation of the allyl aldehyde. Guerbet alcohols are liquid even at low temperatures, and practically cause no skin irritations. It is possible and advantageous to use them as fatting, superfatting, and also as refatting active ingredients in skin care and hair care agents.

The use of Guerbet alcohols in cosmetics is known per se. In most cases, species of this type are characterized by the structure 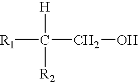
where R₁ and R₂ are normally unbranched alkyl residues.

In accordance with the invention, the Guerbet alcohol or alcohols is/are selected from the group, where
R₁=propyl, butyl, pentyl, hexyl, heptyl, or octyl; and
R₂=hexyl, heptyl, octyl, nonyl, decyl, undecyl, dodecyl, tridecyl, or tetradecyl.

Preferred Guerbet alcohols in accordance with the invention are 2-butyloctanol—it has the chemical structure 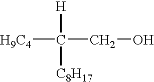
and is available, for example, under the trade name Isofol® 12 from Condea Chemie GmbH—and 2-hexyldecanol—it has the chemical structure 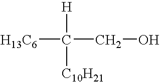
and is available, for example, under the trade name Isofol® 16 from Condea Chemie GmbH. Likewise mixtures of Guerbet alcohols in accordance with the invention are advantageous to use in accordance with the invention. Mixtures of 2-butyloctanol and 2-hexyldecanol are available, for example, under the trade name Isofol® 14 from Condea Chemie GmbH.

The total quantity of Guerbet alcohols in ready-to-use cosmetic or dermatological preparations is advantageously selected from the range up to 25% by weight, preferably 0.5 to 15.0% by weight based on the total weight of the preparations.

It is likewise advantageous to use any mixtures of such oil and wax components for the purposes of the present invention. If need be, it may also be advantageous to use waxes, for example, cetyl palmitate as the only lipid component of the oil phase.

Particularly advantageous medium-polarity lipids for the purposes of the present invention are the substances listed below:

| | | | Polarity |
|---|---|---|---|
| Manufacturer | Trade Name | INCI-Name | [mN/m] |
| Stearinerie Dubois | DUB VCI 10 | Isodecyl neopentanoate | 29.9 |
| Fils | | | |
| ALZO (ROVI) | Dermol IHD | Isohexyl decanoate | 29.7 |
| ALZO (ROVI) | Dermol 108 | Isodecyl octanoate | 29.6 |
| | Dihexyl ether | Dihexyl ether | 29.2 |
| ALZO (ROVI) | Dermol 109 | Isodecyl 3,5,5 trimethyl hexanoate | 29.1 |
| Henkel Cognis | Cetiol SN | Cetearyl isononanoate | 28.6 |
| Unichema | Isopropyl palmitate | Isopropyl palmitate | 28.8 |
| Dow Corning | DC Fluid 345 | Cyclomethicone | 28.5 |
| Dow Corning | Dow Corning Fluid | Cyclopolydimethylsiloxane | 28.5 |
| | 244 | | |
| Nikko Chemicals | Jojoba Oil Gold | | 26.2 |
| Superior Jojoba | | | |
| Oil Gold | | | |
| Wacker | Wacker AK 100 | Dimethicone | 26.9 |
| ALZO (ROVI) | Dermol 98 | 2-Ethylhexanoic acid 3,5,5 trimethyl | 26.2 |
| | | ester | |
| Dow Corning | Dow Corning Fluid | Open | 25.3 |
| | 246 | | |
| ALZO (ROVI) | Dermol 139 | Isotridecyl-3,5,5-trimethyl | 24.5 |
| | | hexanonanoate | |
| | Cegesoft C24 | Octyl palmitate | 23.1 |
| Gattefossé | M.O.D. | Octyldodeceyl myristate | 22.1 |
| | Macadamia Nut | | 22.1 |
| | Oil | | |
| Bayer AG, | Silikonöl VP 1120 | Phenyl trimethicone | 22.7 |
| Dow Corning | | | |
| CONDEA Chemie | Isocarb 12 | Butyl octanoic acid | 22.1 |
| Henkel Cognis | Isopropyl stearate | Isopropyl stearate | 21.9 |
| WITCO, | Finsolv TN | C12-15 Alkyl benzoate | 21.8 |
| Goldschmidt | | | |
| Dr. Straetmans | Dermofeel BGC | Butylene glycol caprylate/caprate | 21.5 |
| Unichema | Miglyol 812 | Caprylic/capric triglyceride | 21.3 |
| Huels | | | |
| Trivent (via S. | Trivent OCG | Tricaprylin | 20.2 |
| Black) | | | |
| ALZO (ROVI) | Dermol 866 | PEG,, Diethyl hexanoate/ | 20.1 |
| | | diisononanoate/ethylhexyl | |
| | | isononanoate | |

Nonpolar oils are, for example, those which are selected from the group of branched and unbranched hydrocarbons and hydrocarbon waxes, in particular Vaseline (petrolatum) paraffin oil, squalane and squalene, polyolefins, and hydrogenated polyisobutenes. Among the polyolefins, the preferred substances are polydecenes.

Particularly advantageous nonpolar lipids for the purposes of the present invention are the substances of the following list:

| | | | Polarity |
|---|---|---|---|
| Manufacturer | Trade Name | INCI-Name | [mN/m] |
| Total SA | Ecolane 130 | Cycloparaffin | 49.1 |
| Neste PAO N.V. | Nexbase 2006 FG | Polydecene | 46.7 |
| (supplier Hansen & Rosenthal) | | | |
| Chemische Fabrik Lehrte | Polysynlane | Hydrogenated | 44.7 |
| | | polyisobutene | |
| Wacker | Wacker Silcone Oil | Polydimethyl siloxane | 46.5 |
| | AK 50 | | |
| EC Erdölchemie (supplier Bayer AG) | Solvent ICH | Isohexadecane | 43.8 |
| DEA Mineralöl (supplier Hansen & | Pionier 2076 | Mineral oil | 43.7 |
| Rosenthal) | | | |
| Tudapetrol | | | |
| DEA Mineralöl (supplier Hansen & | Pionier 6301 | Mineral oil | 43.7 |
| Rosenthal) | | | |
| Tudapetrol | | | |
| Wacker | Wacker Silicone Oil | Polydimethyl siloxane | 42.4 |
| | AK 35 | | |
| EC Erdölchemie GmbH | Isoeicosane | Isoeicosane | 41.9 |
| Wacker | Wacker Silicone Oil | Polydimethylsiloxane | 40.9 |
| | AK 20 | | |
| Condea Chemie | Isofol 1212 | | 40.3 |
| | Carbonate | | |
| Gattefossé | Softcutol O | Ethoxydiglycol oleate | 40.5 |
| Creaderm | Lipodermanol OL | Decyl olivate | 40.3 |
| Henkel | Cetiol S | Dioctylcyclohexane | 39.0 |
| DEA Mineralöl (supplier Hansen & | Pionier 2071 | Mineral oil | 38.3 |
| Rosenthal) | | | |
| Tudapetrol | | | |
| WITCO BV | Hydrobrite 1000 PO | Paraffinum liquidum | 37.6 |
| Goldschmidt | Tegosoft HP | Isocetyl palmitate | 36.2 |
| Condea Chemie | Isofol Ester 1693 | | 33.5 |
| Condea Chemie | Isofol Ester 1260 | | 330 |
| Dow Corning | Dow Corning Fluid | Cyclopentasiloxane | 32.3 |
| | 245 | | |
| Unichema | Prisorine 2036 | Octyl Isostearate | 31.6 |
| Henkel Cognis | Cetiol CC | Dicaprylyl carbonate | 31.7 |
| ALZO (ROVI) | Dermol 99 | Trimethylhexyl | 31.1 |
| | | isononanoate | |
| ALZO (ROVI) | Dermol 89 | 2-Ethylhexyl | 31.0 |
| | | Isononanoate | |
| Henkel Cognis | Cetiol OE | Dicaprylyl ether | 30.9 |
| | Dihexylcarbonate | Dihexyl carbonate | 30.9 |
| Albemarle S.A. | Silkflo 366 NF | Polydecene | 30.1 |
| Unichema | Estol 1540 EHC | Octyl cocoate | 30.0 |

However, it is also advantageous to use mixtures of lipids having a higher and lower polarity and the like. Thus, the oil phase may advantageously be selected from the group of branched and unbranched hydrocarbons and hydrocarbon waxes, the dialkyl ethers, the group of the saturated or unsaturated, branched or unbranched alcohols, as well as the fatty acid triglycerides, in particular the triesters of glycerin of saturated and/or unsaturated, branched and/or unbranched alkanoic acids having a chain length of 8 to 24, in particular 12-18 carbon atoms. The fatty acid triglycerides may advantageously be selected from the group of the synthetic, semisynthetic, and natural oils, olive oil, sunflower seed oil, soybean oil, peanut oil, rape seed oil, almond oil, palm oil, coconut oil, palm kernel oil, and more of the like, provided the conditions of claim 1 are met.

Fat and/or wax components that are advantageous to use in accordance with the invention may be selected from the group of vegetable waxes, animal waxes, mineral waxes and petrochemical waxes. Examples which are favorable according to the invention are candelilla wax, carnauba wax, Japan wax, esparto grass wax, cork wax, guaruma wax, rice germ oil wax, sugarcane wax, berry wax, ouricury wax, montan wax, jojoba wax, shea butter, beeswax, shellac wax, spermaceti, lanolin (wool wax), uropygial grease, ceresin, ozocerite (earth wax), paraffin waxes, and microcrystalline waxes, provided the conditions of claim 1 are met.

Other advantageous fat and/or wax components are chemically modified waxes and synthetic waxes such as, for example, those obtainable under the trade names Syncrowax HRC (glyceryl tribehenate), and Syncrowax AW 1C (C₁₈₋₃₆-fatty acid) from CRODA GmbH, and also montan ester waxes, Sasol waxes, hydrogenated jojoba waxes, synthetic or modified beeswaxes (e.g. dimethicone copolyol beeswax and/or C₃₀₋₅₀-alkyl beeswax), polyalkylene waxes, polyethylene glycol waxes, but also chemically modified fats, such as, for example, hydrogenated vegetable oils (for example, hydrogenated castor oil and/or hydrogenated coconut oil glycerides), triglycerides, such as, for example, trihydroxy stearin, fatty acids, fatty acid esters and glycol esters, such as, for example, C₂₀₋₄₀-alkyl stearate, C₂₀₋₄₀-alkylhydroxystearoyl stearate and/or glycol montanate. Also advantageous are certain organosilicon compounds, which have physical properties similar to the specified fatty and/or wax components, such as, for example, stearoxytrimethylsilane, provided the conditions of claim 1 are met.

In accordance with the invention, the fat and/or wax components may be present both individually and as a mixture. Likewise any desired mixtures of such oil and wax components may be advantageously used for the purposes of the present invention.

Advantageously, the oil phase is selected from the group of 2-ethylhexyl isostearate, octyldodecanol, isotridecyl isononanoate, butylene glycol, dicaprylate/dicaprate, 2-ethylhexyl cocoate, C₁₂₋₁₅-alkyl benzoate, caprilic-capric acid triglyceride, dicaprylyl ether, provided the conditions of claim 1 are met.

Especially advantageous are mixtures of octyldodecanol, carpylic-capric acid triglyceride, dicaprylyl ether, dicaprylyl carbonate, coconut oil glycerides, or mixtures of C₁₂₋₁₅-alkyl benzoate and 2-ethylhexyl isostearate, mixtures of C₁₂₋₁₅-alkyl benzoate and butylene glycol dicaprylate/dicaprate, as well as mixtures of C₁₂₋₁₅-alkyl benzoate, 2-ethylhexyl isostearate, and isotridecyl isononanoate, provided the conditions of claim 1 are met.

Of the hydrocarbons, paraffin oil, cycloparaffin, squalane, squalene, hydrogenated polyisobutene, or polydecene are advantageous to use for the purposes of the present invention, provided the conditions of claim 1 are met.

Silicones

It is also possible and advantageous to select the oil phase of the preparations according to the invention in part or in full from the group of cyclic and/or linear silicones, which are also named “silicone oils” within the scope of the present disclosure. Such silicones or silicone oils may be present as monomers, which are normally characterized by structural elements as follows:

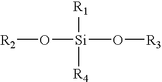

Silicone oils are high-molecular, synthetic polymeric compounds, in which silicon atoms are linearly linked and/or crosslinked via oxygen atoms, and the residual valences of the silicon are saturated by hydrocarbon residues (in most cases methyl-, to a lesser extent ethyl-, propyl-, phenyl groups, and others).

Linear silicones with a plurality of siloxyl units that are advantageous to use in accordance with the invention, are generally characterized by structural elements, such as 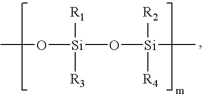
where the silicon atoms can be substituted with the same or different alkyl residues and/or aryl residues, which are generally shown by the residues R₁-R₄ (i.e., the number of the different residues is not necessarily limited to as many as four); m may have values from 2 to 200,000.

Systematically, the linear silicone oils are called polyorganosiloxanes; the methyl-substituted polyorganosiloxanes, which represent in terms of quantity the most important compounds of this group and are characterized by the following structural formula 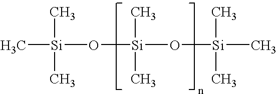
are also named polymethylsiloxanes or dimethicones (INCI). Dimethicones exist in different chain lengths or with different molecular weights. Dimethicones of different chain lengths and phenyltrimethicones are particularly advantageous linear silicone oils for the purposes of the present invention.

Particularly advantageous polyorganosiloxanes for the purposes of the present invention are furthermore, for example, dimethyl polysiloxanes [poly(dimethylsiloxane)], which are available, for example, under the tradenames ABIL 10 to 10,000 from Th. Goldschmidt. Also advantageous are phenylmethyl polysiloxanes (INCI: phenyl dimethicone, phenyl trimethicone), cyclic silicones (octamethyl cyclotetrasiloxane or decamethyl cyclopentasiloxane), which are also named according to INCI cyclomethicones, amino-modified silicones (INCI: amodimethicones), and silicone waxes, for example, polysiloxane-polyalkylene copolymers (INCI: stearyl dimethicone and cetyl dimethicone), and dialkoxy dimethylpolysiloxanes (stearoxy dimethicone and behenoxy stearyl dimethicone), which are available as different Abil wax types from Th. Goldschmidt.

Especially advantageous for the purposes of the present invention are also the following listed silicone oils:

| Manu- | | | Polarity |
|---|---|---|---|
| facturer | Trade Name | INCI-Name | [mN/m] |
| Wacker | Wacker Silicone Oil AK | Polydimethylsiloxane | 26.9 |
| | 100 | | |
| Wacker | Wacker Silicone Oil AK 50 | Polydimethylsiloxane | 46.5 |
| Wacker | Wacker Silicone Oil AK 35 | Polydimethylsiloxane | 42.4 |
| Wacker | Wacker Silicone Oil AK 20 | Polydimethylsiloxane | 40.9 |
| Dow | Dow Corning Fluid 245 | Cyclopentasiloxane | 32.3 |
| Corning | | | |
| Dow | Dow Corning Fluid 345 | Cyclomethicone | 28.5 |
| Corning | | | |

Cyclic silicones that are advantageous to use in accordance with the invention are generally characterized by structural elements, as follows: 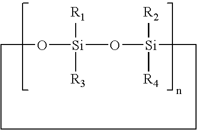
where the silicon atoms can be substituted with the same or with different alkyl residues and/or aryl residues, which are generally represented by the residues R₁-R₄ (i.e., the number of the different residues is not necessarily limited to as many as four); n may assume values from 3/2 to 20. Fractional values for n take into account that uneven numbers of siloxyl groups can be present in the cycle.

Particularly advantageous cyclic silicone oils for the purposes of the present invention are cyclomethicone, in particular cyclomethicone D5 and/or cyclomethicone D6.

Advantageous silicone oils or silicone waxes for the purposes of the present invention are cyclic and/or linear silicone oils and silicone waxes.

It is especially advantageous for the purposes of the present invention to choose a ratio of lipids to silicone oils of approximately 1:1 (generally x:y).

It is advantageous to select phenyl trimethicone as silicone oil. Likewise other silicone oils, for example, dimethicone, phenyl dimethicone, cyclomethicone (octamethyl cyclotetrasiloxane), for example, hexamethyl cyclotrisiloxane, polydimethylsiloxane, poly(methyl)phenylsiloxane) cetyl dimethicone, behenoxy dimethicone are advantageous to use for the purposes of the present invention.

Also advantageous are mixtures of cyclomethicone and isotridecyl isononanoate, as well as those of cyclomethicone and 2-ethylhexyl isostearate.

However, it is also advantageous to choose silicone oils of a constitution similar to that of the foregoing compounds, whose organic side chains are derivatized, for example, polyethoxylated and/or polyproxylated. Among those are, for example, polysiloxane-polyalkyl-polyether copolymers, such as cetyl-dimethicone-copolyol, as well as cetyl-dimethicone-copolyol (and) polyglyceryl-4-isostearate (and) hexyllaurate.

The emulsions of the invention may advantageously contain moisturizing or humectant agents (so-called moisturizers). Advantageous moisturizers for the purposes of the present invention are, for example, glycerin, lactic acid and/or lactate, in particular, sodium lactate, butylene glycol, propylene glycol, biosaccharide gum-1, glycine soya, ethylhexyloxyglycerin, pyrrolidone carboxylic acid, and urea. Furthermore, it is of special advantage to use polymeric moisturizers from the group of the water-soluble and/or water-swellable and/or water-gellable polysaccarides. Particularly advantageous are, for example, hyaluronic acid, chitosan, and/or a fucose-rich polysaccharide, which is filed in the Chemical Abstracts under the Registry Number 178463-23-5, and which can be obtained, for example, under the name Fucogel® 1000 from SOLABIA S.A.

Antioxidants are advantageously selected from the group consisting of amino acids (for example, glycine, lysine, arginine, cysteine, histidine, tyrosine, tryptophan) and derivatives thereof (as salt-, ester-, ether-, sugar-, nucleotide-, nucleoside-, peptide-, and lipid compounds); imidazoles (for example, urocanic acid) and their derivatives (as salt-, ester-, ether-, sugar-, nucleotide-, nucleoside-, peptide-, and lipid compounds); peptides, such as D,L-carnosine, D-carnosine, L-carnosine, anserine, and derivatives thereof (for example, as salt-, ester-, ether-, sugar-, thiol-, nucleotide-, nucleoside-, peptide-, and lipid compounds); carotenoids, carotene (for example, α-carotene, β-carotene, ψ-lycopene, phytoene) and derivatives thereof (for example, as salt-, ester-, ether-, sugar-, nucleotide-, nucleoside-, peptide-, and lipid compounds); chlorogenic acid and its derivates (as salt-, ester-, ether-, sugar-, thiol-, nucleotide-, nucleoside-, peptide-, and/or lipid compounds); aurothioglucose, propylethiouracil and other thiols (for example, thioredoxin, lipoic acid, glutathione, cysteine, cystine, cystamine, and glycosyl-, N-acetyl-, methyl-, ethyl-, propyl-, amyl-, butyl- and lauryl-, palmitoyl-, oleyl-, γ-linoleyl-, cholesteryl-, and glyceryl esters thereof, as well as their salts, dilauryl thiodipropionate, distearyl thiodipropionate, thiodipropionic acid and derivatives thereof (as salt-, ester-, ether-, sugar-, thiol-, nucleotide-, nucleoside-, peptide and/or lipid compounds), and sulfoximine compounds (for example, homocysteine sulfoximine, buthionine sulfones, penta-, hexa-, heptathionine sulfoximine) in very low tolerated doses (for example, pmol to μmol/kg). Furthermore, metal chelating agents (for example, apoferritin, desferral, lactoferrin, α-hydroxy fatty acids, palmitic acid, phytic acid), and derivatives thereof (as salt-, ester-, ether-, sugar-, thiol-, nucleotide-, nucleoside-, peptide and/or lipid compounds); α-hydroxy acids (for example, citric acid, lactic acid, malic acid), humic acid, bile acid, bile extracts, bilirubin, biliverdin, EDTA, EGTA and derivatives thereof; unsaturated fatty acids and derivatives thereof; unsaturated fatty acids and their derivatives (for example, γ-linolenic acid, linoleic acid, oleic acid), folic acid, and derivatives thereof; furfurylidene sorbitol and its derivatives; ubiquinone, ubiquinol, plastoquinone and their derivatives (as salt-, ester-, ether-, sugar-, thiol-, nucleotide-, nucleoside-, peptide and lipid compounds); vitamin C and derivatives (for example, ascorbyl palmitate, Mg-ascorbyl phosphate, ascorbyl acetate); tocopherols and derivatives thereof (for example, vitamin E acetate) as well as phenolic compounds and plant extracts containing same, such as, for example, flavonoids (for example, glycosylrutin, ferulic acid, caffeic acid); furfurylidene glucitol, butylhydroxytoluene, butylhydroxyanisole, nordihydroguaiac resin acid; nordihydroguaiaretic acid; trihydroxybutyrophenone and derivatives thereof (as salt-, ester-, ether-, sugar-, nucleotide-, nucleoside-, peptide and lipid compounds); uric acid and its derivatives; mannose and its derivatives (as salt-, ester-, ether-, sugar-, nucleotide-, nucleoside-, peptide and lipid compounds); zinc and its derivatives (for example, ZnO, ZnSO₄); selenium and its derivatives (for example, selenium methionine, Ebselen); stilbene and its derivatives for example, stilbene oxide, trans-stilbene oxide); and derivatives (salt-, ester-, ether-, sugar-, thiol-, nucleotide-, nucleoside, peptide-, and/or lipid compounds) of these referenced active ingredients, which are suitable according to the invention.

Further advantageous active ingredients for the purpose of the present invention are natural ingredients and/or their derivatives, such as, for example, alpha-lipoic acid, phytoene, lycopene, D-biotin, coenzyme Q10, carnitine, vitamin A and its derivatives, natural and/or synthetic isoflavonoids (for example, genistein, daidzein), creatine, creatinine, lignans, taurine, and/or beta-alanine.

Formulations of the invention, which contain, for example, known antiwrinkle agents, such as flavone glycosides (in particular alpha-glycosylrutin), coenzyme Q10, vitamin E and/or derivatives, and the like, are especially suited for prophylaxis and treatment of cosmetic or dermatological skin changes, as they occur, for example, in the case of skin ageing (such as, for example, dryness, roughness, and formation of dryness lines, itching, reduced refatting (for example, after washing), visible vascular dilations (teleangiectases, cuperosis), flaccidity and formation of wrinkles and lines, local hyperpigmentation, hypopigmentation, abnormal pigmentation (for example, age spots), increased susceptibility to mechanical stress (for example, cracking), and the like. They are furthermore suited for treating the appearance of dry and rough skin.

According to the invention, however, it is also possible and advantageous to incorporate in the preparations of the invention other pharmaceutically or dermatologically active substances, such as, for example, skin-soothing and skin-caring substances. Among those are, for example, panthenol, allantoin, tannin, antihistamines, antiphlogistic agents, glucocorticoids (for example, hydrocortisone), as well as plant ingredients, such as azulene and bisabolol, glycyrrhizin, hamamelin, and plant extracts, such as chamomile, aloe vera, hamamelis, and the root of licorice. Likewise, the vitamin D₃ analogues tacalcitol, calcipotriol, cholecalciferol, as well as calcitrol (vitamin D₃), and/or esters of the fumaric acid may be successfully included in the preparations.

The preparations of the invention may contain one or more of these active constituents (moisturizers, antioxidants, other active ingredients), each in a concentration of 0.001 to 30% by weight based on the total weight of the preparation.

It is advantageous in accordance with the invention, when the emulsions of the invention include a content of UV protective substances. For example, day creams or makeup products normally include UV-A or UV-B filter substances. Likewise UV protective substances constitute in the same way as antioxidants and, if desired, preservatives, an effective protection of the preparations against deterioration. Also favorable are cosmetic and dermatological preparations, which are present in the form of a sunscreen.

Accordingly, the emulsions contain for the purposes of the present invention preferably at least one UV-A- and/or UV-B filter substance. While not necessary, the formulations may also contain one or more organic and/or inorganic pigments as UV-B filter substances, which may be present in the water- and/or oil phase.

Preferred inorganic pigments are metal oxides and/or other metal compounds that are difficult to dissolve or insoluble in water, in particular the oxides of titanium (TiO₂), zinc (ZnO), iron (for example, Fe₂O₃), zirconium (ZrO₂), silicon (SiO₂), manganese (for example, MnO), aluminum (Al₂O₃), cerium (for example (Ce₂O₃), mixed oxides of the corresponding metals, as well as mixtures of such oxides, as well as the sulfate of barium (BASO₄).

The titanium dioxide pigments may be present in the crystalline modification rutile as well as anatase. For the purposes of the present invention, they may advantageously be surface treated (“coated”). In this process, it is intended to form or maintain, for example, a hydrophilic, amphiphilic, or hydrophobic character. This surface treatment may consist in that the pigments are provided by methods known per se with a thin hydrophilic and/or hydrophobic, inorganic and/or organic coating. For the purposes of the present invention, the different surface coatings may also contain water.

The foregoing coated or uncoated titanium dioxides may also be used for the purposes of the present invention in the form of commercially available, oily or aqueous predispersions. It is possible and advantageous to add to these predispersions dispersants and/or solubilization agents.

The titanium dioxides of the invention are characterized by a primary particle size from 10 nm to 150 nm.

| | | Additional | |
|---|---|---|---|
| | | Constituents of the | |
| Trade name | Coating | Predispersion | Manufacturer |
| MT-100TV | Aluminumhydroxide | — | Tayca Corporation |
| | stearic acid | | |
| MT-100Z | Aluminumhydroxide | — | Tayca Corporation |
| | stearic acid | | |
| MT-100F | Stearic acid | — | Tayca Corporation |
| | iron oxide | | |
| MT-500SAS | Alumina, silica | — | Tayca Corporation |
| | silicone | | |
| MT-100AQ | Silica | — | Tayca Corporation |
| | aluminumhydroxide | | |
| | alginic acid | | |
| Eusolex T-2000 | Alumina | — | Merck KgaA |
| | simethicone | | |
| Eosolex TS | Alumina, stearic acid | — | Merck KgaA |
| Titanium dioxide P25 | None | — | Degussa |
| Titanium dioxide | Octyltrimethylsilane | — | Degussa |
| T805 | | | |
| (Uvinul TiO₂) | | | |
| UV-Titanium X170 | Alumina | — | Kemira |
| | dimethicone | | |
| UV-Titan X161 | Alumina, silica | — | Kemira |
| | stearic acid | | |
| Tioveil AQ 10PG | Alumina | Water | Solaveil |
| | silica | propylene glycol | Uniquema |
| Mirasun TiW 60 | Alumina | Water | Rhone-Poulenc |
| | silica | | |

For the purposes of the present invention, specially preferred titanium dioxides are MT-100 Z and MT-100 TV from Tayca Corporation, Eusolex T-2000 and Eusolex TS from Merck, and titanium dioxide T 805 from Degussa.

Zinc oxides for the purposes of the present invention may also be used in the form of commercially available, oily or aqueous predispersions. In accordance with the invention, suitable zinc oxide particles and predispersions of zinc oxide particles are characterized by a primary particle size of <300 nm, and are available from the listed manufacturers under the following trade names:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |
| MZ 707M | 7% Dimethicone | M. Tayca Corp. |
| Nanox 500 | / | Elementis |
| ZnO Neutral | / | H&R |

Specially preferred zinc oxides for the purposes of the present invention are Z-Cote HP1 from BASF and the zinc oxide NDM from Haarmann & Reimer.

The total quantity of one or more inorganic pigments in the finished cosmetic preparation is advantageously selected from a range of 0.1% to 25% by weight, preferably 0.5% to 18% by weight.

An advantageous organic pigment for the purposes of the present invention is 2,2′-methylene-bis-(6-(2H-benzotriazole-2-yl)-4-(1,1,3,3-tetramethylbutyl)-phenol) [INCI: bisoctyltriazole], which is available from CIBA-Chemikalien GmbH under the trade name Tinosorb® M.

Advantageous UV-A filter substances for the purposes of the present invention are dibenzoylmethane derivatives, in particular 4-(ter.-butyl)-4′-methoxydibenzoylmethane (CAS No. 70356-09-1), which is sold by Givaudan under the trademark Parsol® 1789 and by Merck under the trade name Eusolex® 9020.

Further advantageous UV-A filter substances are hydroxybenzophenones, which are characterized by the following structural formula: 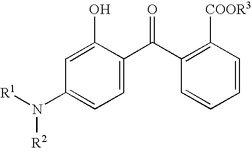
where
R¹ and R are independently of each other hydrogen, C₁-C₂₀-alkyl, C₃-C₁₀-cycloalkyl, or C₃-C₁₀-cycloalkenyl, with the substituents R¹ and R² being able to form a 5- or 6-member ring together with the nitrogen atom to which they are linked, and
R³ is a C₁-C₂₀ alkyl residue.

A particularly advantageous hydroxybenzophenone for the purposes of the present invention is 2-(4′-diethylamino-2′-hydroxybenzoyl)benzoic acid hexyl ester (also known as: aminobenzophenone), which is available from BASF under the trade name Uvinul A Plus.

Advantageous UV filter substances for the purposes of the present invention further include so-called broadband filters, i.e. filter substances which absorb both UV-A and UV-B radiation.

Advantageous broadband filters or UV-B filter substances are, for example, bis-resorcinyltriazine derivatives with the following structure: 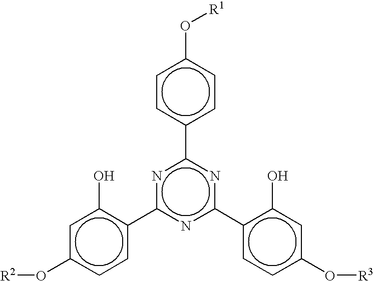
where R¹, R², and R³ are selected independently of one another from the group of branched and unbranched alkyl groups having 1 to 10 carbon atoms, or represent an individual hydrogen atom. Especially preferred is 2,4-bis{[4-(2-ethyl-hexyloxy)-2-hydroxy]phenyl}-6-(4-methoxyphenyl)-1,3,5-triazine (INCI: Aniso triazine), which is available from CIBA-Chemikalien GmbH under the trade name Tinosorb® S.

Especially advantageous preparations for the purposes of the present invention, which are characterized by a high or very high UV-A protection, preferably contain a plurality of UV-A- and broadband filters, in particular dibenzoylmethane derivatives [for example, 4-(tert.-butyl)-4′-methoxydibenzoylmethane], benzotriazole derivatives [for example, 2,2′-methylene-bis-(6-(2H-benzotriazole-2-yl)-4-(1,1,3,3-tetramethylbutyl)-phenol)], phenylene-1,4-bis(2-benzimidazyl)-3,3′-5,5′-tetrasulfonic acid and/or its salts, 1,4-d(2-oxo-10-sulfo-3-bornylidenemethyl)-benzene and/or the salts thereof, and/or 2,4-bis{[4-(2-ethyl-hexyloxy)-2-hydroxy]phenyl}-6-(4-methoxyphenyl)-1,3,5-triazine, each individually or in any combinations with one another.

Likewise other UV filter substances, which have the structural motif 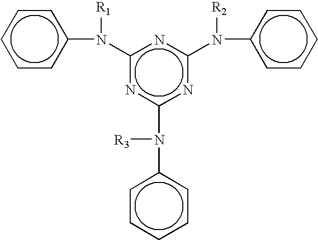
are advantageous filter substances for the purposes of the present invention, for example, s-triazine derivatives as disclosed in EP 570 838 A1, whose chemical structure is represented by the generic formula 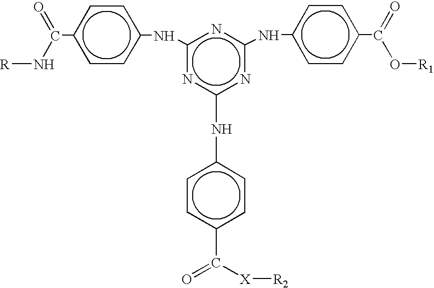
where
R is a branched or unbranched C₁₋₁₈ alkyl residue, a C₅₋₁₂ cycloalkyl residue, if need be, substituted with one of more C₁₋₄ alkyl groups,
X is an oxygen atom or an NH group, and
R₁ is a branched or unbranched C₁₋₁₈ alkyl residue, a C₅₋₁₂ cycloalkyl residue, if need be, substituted with one of more C₁₋₄ alkyl groups, or a hydrogen atom, an alkaline metal, an ammonium group, or a a group of the formula 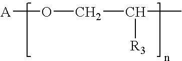
   where
   A is a branched or unbranched C₁₋₁₈ alkyl residue, a C₅₋₁₂ cycloalkyl or aryl residue, if need be, substituted with one of more C₁₋₄ alkyl groups,
   R₃ is a hydrogen atom or a methyl group, and
   n is a number from 1 to 10;
R₂ is a branched or unbranched C₁₋₁₈ alkyl residue, a
   C₅₋₁₂ cycloalkyl residue, if need be, substituted with one of more C₁₋₄ alkyl groups, when X is the NH group, and
   a branched or unbranched C₁₋₁₈ alkyl residue, a C₅₋₁₂ cycloalkyl residue, if need be, substituted with one of more C₁₋₄ alkyl groups, or a hydrogen atom, an alkaline metal atom, an ammonium group or a group of the formula 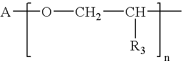
   where
   A is a branched or unbranched C₁₋₁₈ alkyl residue, a C₅₋₁₂ cycloalkyl residue, if need be, substituted with one of more C₁₋₄ alkyl groups,
   R₃ is a hydrogen atom or a methyl group, and
   n is a number from 1 to 10,
   when X is an oxygen atom.

Furthermore, a very preferred UV filter substance for the purposes of the present invention is an asymmetrically substituted s-triazine, which is also referred to in the following as dioctylbutylamidotriazone (INCI: Dioctyl butamido triazone) and can be obtained from Sigma 3V under the trade name UVASORB HEB.

Advantageous for the purposes of the present invention is also a symmetrically substituted s-triazine, i.e., 4,4′,4″-(1,3,5-triazine-2,4,6-triyltriimino)-tris-benzoic acid-tris(2-ethylhexyl-ester), synonym: 2,4,6-tris-[anilino-(p-carbo-2′-ethyl-1′-hexyloxy)]-1,3,5-triazine (INCI: Octyl Triazone), which BASF Aktiengesellschaft markets under its trade name UVINUL® T 150.

Likewise EP 775 698 discloses bis-resorcinyl triazine derivatives that are to be used with preference and whose chemical structure is represented by the generic formula 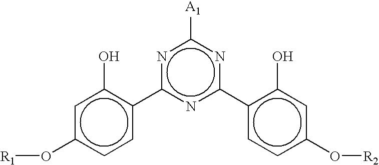
where R₁, R₂, and A₁ represent a great variety of organic residues.

Also advantageous for the purposes of the present invention are 2,4-bis-{[4-(3-sulfonato)-2-hydroxy-propyloxy)-2-hydroxy]-phenyl}-6-(4-methoxyphenyl)-1,3,5-triazine sodium salt, 2,4-bis-{[4-(3-(2-propyloxy)-2-hydroxy-propyloxy)-2-hydroxy]-phenyl}-6-(4-methoxyphenyl)-1,3,5-triazine, 2,4-bis-{[4-(2-ethyl-hexyloxy)-2-hydroxy]-phenyl}-6-[4-(2-methoxyethyl-carboxyl)-phenylamino]-1,3,5-triazine, 2,4-bis-{[4-(3-(2-propyloxy)-2-hydroxy-propyloxy)-2-hydroxy]-phenyl}-6-[4-(2-ethyl-carboxyl)-phenylamino]-1,3,5-triazine, 2,4-bis-{[4-(2-ethyl-hexyloxy)-2-hydroxy]-phenyl}-6-(1-methyl-pyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2″-methylpropenyloxy)-2-hydroxy]-phenyl}-6-(4-methoxyphenyl)-1,3,5-triazine, and 2,4-bis-{[4-(1′,1′,1′,3′,5′,5′,5′-heptamethylsiloxy-2″-methyl-propyloxy)-2-hydroxy]-phenyl}-6-(4-methoxyphenyl)-1,3,5-triazine.

An advantageous broadband filter for the purposes of the present invention is 2,2′-methylene-bis-(6-(2H-benzotriazole-2-yl)-4-(1,1,3,3-tetramethylbutyl)-phenol), which is available from CIBA-Chemikalien GmbH under the trade name Tinosorb® M.

A further advantageous broadband filter for the purposes of the present invention is 2-(2H-benzotriazole-2-yl)-4-methyl-6-[2-methyl-3-[1,3,3,3-tetramethyl-1-[(trimethylsilyl)oxy]disiloxanyl]propyl]-phenol (CAS-Nr.: 155633-54-8) under the INCI name Drometrizole Trisiloxane.

The UV-B and/or broadband filters may be oil-soluble or water-soluble. Advantageous oil-soluble UV-B filter and/or broadband filter substances are, for example:
derivatives of 3-benzylidene camphor, preferably 3-(4-methylbenzylidene) camphor, 3-benzylidene camphor;
derivatives of 4-aminobenzoic acid, preferably 2-(ethylhexyl)₄-dimethylamino-benzoate, amyl 4-(dimethylamino)-benzoate;
2,4,6-trianilino-(p-carbo-2′-ethyl-1′-hexyloxy)-1,3,5-triazine;
esters of benzalmalonic acid, preferably di(2-ethylhexyl)4-methoxybenzalmalonate;
esters of cinnamic acid, preferably (2-ethylhexyl)4-methoxy cinnamate, isopentyl 4-methoxy cinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone; as well as
polymer-linked UV filters.

For the purposes of the present invention, especially advantageous liquid UV filter substances that can be used at room temperature are homomenthyl salicylate (INCI: homosalate), 2-ethylhexyl-2-hydroxy benzoate (2-ethylhexyl salicylate, octyl salicylate, INCI: octyl salicylate), 4-isopropylbenzyl salicylate und esters of the cinnamic acid, preferably 4-methoxy cinnamic acid(2-ethylhexyl)ester (2-ethylhexyl-4-methoxycinnamate, INCI: octyl methoxycinnamate), and 4-methoxycinnamic acid-isopentylester (isopentyl-4-methoxycinnamate, INCI: isoamyl p-methoxycinnamate), 3-(4-(2,2-bis-ethoxycarbonylvinyl)-phenoxy)propenyl)-methoxysiloxane/ dimethylsiloxane copolymer (INCI: dimethicodiethylbenzalmalonate), which is available, for example, under the trade name Parsol® SLX from Hoffmann La Roche.

A further light protective filter substance that is advantageous to use in accordance with the invention, is ethylhexyl-2-cyano-3,3-diphenylacrylate (octocrylene), which is available from BASF under the name Uvinul® N539.

It can also be of considerable advantage to use polymer-linked and polymeric UV filter substances in preparations according to the present invention, in particular such as are disclosed in WO-A-92/20690.

The list of the specified UV filters that can be used for the purposes of the present invention is naturally not intended to be limiting.

Advantageously, the preparations of the invention contain the substances, which absorb UV radiation in the UV-A and/or UV-B range, where the total amount of the filter substances is, for example, from 0.1% to 30% by weight, preferably 0.5% to 20% by weight, in particular 1.0% to 15.0% by weight based on the total weight of the preparations, for the purpose of making available cosmetic preparations, which protect hair or skin against the entire range of the ultraviolet radiation. They can also serve as sunscreens for the hair or skin.

To improve the water resistance of cosmetic and dermatological light protective preparations, it can be of advantage to incorporate film formers in the cosmetic or dermatological preparations. To this end, suitable film formers of the art are, for example, polyurethanes (for example, the Avalure® types from Goodrich), dimethicone copolyol polyacrylate (Silsoft Surface® from Witco Organo Silicones Groups). PVP/VA (VA=vinyl acetate) copolymer (Luviscol VA 64 powder from BASF), C₂₀₋₄₀ carbonic acid with polyethylene (Performacid 350 from New Phase Technologies), as well as film formers from the group of polymers on the basis of polyvinyl pyrrolidone (PVP): 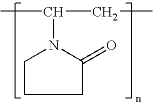
It is especially preferred to use copolymers of polyvinyl pyrrolidone, for example, PVP hexadecene copolymer and PVP eicosene copolymer, which are available under the trade names Antaron V216 and Antaron V220 from GAF Chemicals Corporation, as well as Tricotayl PVP, and more of the like.

According to the invention, the emulsion of the invention may advantageously contain microparticles, whose an average particle diameter is between 1 nm and 200 nm, very advantageously between 5 nm and 100 nm.

It is furthermore advantageous to choose the concentration of all particles greater than 0.1% by weight, very advantageously between 0.1% and 30% by weight based on the total weight of the preparations.

For the present invention it is essentially irrelevant, in which of the possibly naturally occurring modifications the particles are present.

It is preferred to use untreated, almost pure pigment particles, in particular those, which can also be used as dyes in the food industry and/or as absorbers of UV radiation in sunscreens. Advantageous are, for example, the zinc oxide pigments as are available from Merck, as well as those, which can be obtained under the trade names Zinc Oxide neutral from Haarmann & Reimer or NanoX from the Harcos Chemical Group.

Also advantageous are pigments, which are made water-repellent (“coated”) on their surface. This surface treatment may consist in providing the pigments with a thin hydrophobic coating by methods known per se.

Especially advantageous are TiO₂ pigments, for example, those which are coated with aluminum stearate and can be obtained under the trade name MT 100 T from TAYCA.

A further advantageous coating of the inorganic pigments consists of dimethyl polysiloxane (also: dimethicone), a mixture of fully methylated, linear siloxane polymers, which are terminally blocked with trimethylsiloxy units. Particularly advantageous for the purposes of the present invention are zinc oxide pigments, which are coated in this way.

Advantagous is also a coating of the inorganic pigments with a mixture of dimethyl polysiloxane, in particular dimethyl polysiloxane with an average chain length of 200 to 350 dimethyl polysiloxane units, and silica gel, which is also known as simethicone. It is in particular of advantage, when the inorganic pigments are coated in addition with aluminum hydroxide or hydrous aluminum oxide (also: Alumina, CAS No. 1333-84-2). Particularly advantageous are titanium dioxides, which are coated with simethicone and alumina. In this instance, the coating may also contain water. An example therefor is titanium dioxide that can be obtained from Merck under the tradename Eusolex T2000.

Advantageous for the purposes of the present invention is furthermore the use of a mixture of different pigment types both within a crystal, for example, as a ferrous mixed oxide, and by combining a plurality of pigment types within a preparation.

Preferred are furthermore boron nitride particles, for example boron nitrides of the following list:

| Trade Name | Available from |
|---|---|
| Boron Nitride Powder | Advanced Ceramics |
| Boron Nitride Powder | Sintec Keramic |
| Ceran Blanche | Kawasaki |
| HCST Boron Nitride | Stark |
| Très BN ® | Carborundum |
| Wacker-Boron Nitride BNP | Wacker-Chemie |

Wacker-Boron Nitride BNP Wacker-Chemie

It is advantageous to choose the average particle diameter of the used boron nitride particles smaller than 20 μm, very advantageously smaller than 15 μm.

Advantageous are furthermore the boron nitride particles that are available from Carborundum under the trade name Très BN® UHP 1106, and which are treated with dimethicone.

Advantageous is furthermore a coating of the boron nitride particles with polymethylhydrogensiloxane, a linear polysiloxane also known as methicone. Advantageous, methicone-treated boron nitride particles are, for example, those which are available from Carborundum under the trade name Très BN® UHP 1107.

Further advantageous pigments are microfine polymer particles.

Advantageous microfine polymer particles are for the purposes of the present invention, for example, polycarbonate, polyether, polyethylene, polypropylene, polyvinylchloride, polystyrene, polyamide, and more of the like.

Advantageous in accordance with the invention are, for example, microfine polyamide particles, which are available under the trade name SP-500 from TORAY. Also advantageous are particles of polyamide 6 (also: Nylon 6) or polyamide 12 (also: Nylon 12). Polyamide 6 is the polyamide produced from ε-aminocaproic acid (6-aminohexanoic acid) or ε-caprolactam [poly(ε-caprolactam)], and polyamide 12 is a poly(ε-laurinlactam) from ε-laurinlactam. Advantageous for the purposes of the present invention are, for example, Orgasol® 1002 (polyamide 6) and Orgasol® 2002 (polyamide 12) from ELF ATOCHEM.

While not required, it is particularly advantageous to coat the used microfine polymer particles on their surface. This surface treatment may consist in providing the pigments with a thin hydrophilic coating by methods known per se. Advantageous coatings consist, for example, of TiO₂, ZrO₂, or also other polymers.

Particularly advantageous microfine polymer particles for the purposes of the present invention are furthermore available by the method for providing hydrophobic polymer particles with a hydrophilic coating, as is disclosed in U.S. Pat. No. 4,898,913.

It is advantageous to select the average particle diameter of the used microfine polymer particles smaller than 100 μm, very advantageously smaller than 50 μm. In this connection, the form (platelets, rodlets, pellets), in which the used polymers are present, does essentially not matter.

It is furthermore advantageous to use modified polysaccharides.

Modified polysaccharides for the purposes of the present invention can be obtained, for example, by converting starch with mono-, bi-, or polyfunctional reagents or oxidation agents in reactions that largely proceed in a polymer-analogous way.

Reactions of this type are largely based on transforming the hydroxy groups of polyglucans by etherification, esterification, or selective oxidation. In this process, for example, so-called starch ethers and starch esters of the general structural formula develop 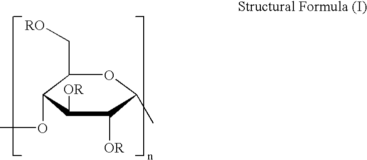
where R can be, for example, a hydrogen and/or an alkyl, and/or an aryl residue (in the case of starch ether), or a hydrogen, and/or an organic, and/or an inorganic acid residue (in the case of starch ester). Starch ethers and starch esters are advantageous modified polysaccharides for the purposes of the present invention.

Especially advantageous starch ethers are, for example, those which can be obtained by etherifying starch with tetramethylol acetylene diurea, and which are referred to as Amylym non mucilaginosum (nonswelling starch).

Especially advantageous are also starch esters and their salts, for example, the sodium- and/or aluminum salts of low-substituted half-esters of the starch, in particular sodium starch n-octenyl succinate of the structural formula (I), where R is characterized by the following structure 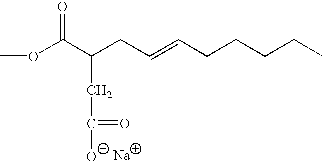
and which is available, for example, under the trade name Amiogum® 23 from CERESTAR, as well as aluminum starch octenyl succinates, in particular those which are available under the trade names Dri Flo® Elite LL, and Dri Flo® PC from CERESTAR.

It is advantageous to choose the average particle diameter of the used modified polysaccharides smaller than 20 μm, very advantageously smaller than 15 μm.

The list of the referenced modified polysaccharides is naturally not intended to be limiting. Modified polysaccharides for the purposes of the present invention are available in numerous ways of both chemical and physical nature, which are known per se.

According to the invention, the compositions may contain besides the foregoing substances, additional substances as are customary in cosmetics, for example, perfume, dyes, antimicrobial agents, refatting agents, complexing and sequestering agents, pearlescent agents, plant extracts, vitamins, active agents, preservatives, bactericides, surfactants, pigments, which have a coloring effect, thickeners, softening, moisturizing, and/or humectant substances, or other common constituents of a cosmetic or dermatological formulation, such as alcohols, polyols, polymers, foam stabilizers, electrolytes, organic solvents, or silicone derivatives.

In accordance with the invention, the preparation of the invention may advantageously contain one or more preservatives. Advantageous preservatives for the purposes of the present invention are, for example, formaldehyde donors (such as, for example, DMDM hydantoin, which is available under the trade name Glydant® from Lonza), iodopropyl butylcarbamates (for example, those which are available under the trade names Glycacil-S from Lonza and/or Dekaben LMB from Jan Dekker), parabens (i.e., alkyl esters of the p-hydroxybenzoic acid, such as methyl-, ethyl-, propyl-, and/or butylparaben), phenoxyethanol, ethanol, benzoic acid, and more of the like. Normally, the preservation system furthermore comprises in accordance with the invention advantageously preservative auxiliaries, such as, for example octoxyglycerin, glycine soya, etc. The following table gives an overview of some preservatives that are advantageous in accordance with the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na |
| | salt |
| E 216 | n-Propyl p-hydroxybenzoate |
| E 217 | n-Propyl p-hydroxybenzoate Na |
| | salt |
| E 218 | Methyl p-hydroxybenzoate |
| E 219 | Methyl p-hydroxybenzoate Na |
| | salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite |
| E 230 | Biphenyl (diphenyl) |
| E 231 | Orthophenylphenol |
| E 232 | Sodium |
| | orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrate |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable according to the invention are preservatives or preservative auxiliaries that are customary in cosmetics, such as dibromocyanobutane (2-bromo-2-bromomethylglutarodinitrile), 3-iodo-2-propynyl butylcarbamate, 2-bromo-2-nitropropane-1,3-diol, imidazolidinyl urea, 5-chloro-2-methyl-4-isothiazolin-3-one, 2-chloroacetamide, benzalkonium chloride, and benzyl alcohol.

According to the invention, it is especially preferred to use as preservatives benzoic acid, and/or salicylic acid, and/or their derivatives or salts.

According to the invention, it is advantageous that one of the two partial preparations or both partial preparations contain one or more preservatives in a concentration of 2% by weight or less than 2% by weight, preferably 1.5% by weight, or less than 1.5% by weight, and more preferably 1% by weight or less than 1% by weight, each based on the total weight of the preparation A or B.

The preparations of the invention contain in an advantageous manner one or more conditioners. Preferred conditioners in accordance with the invention are, for example, all compounds which are listed in the International Cosmetic Ingredient Dictionary and Handbook (Volume 4, Publishers: R. C. Pepe, J. A. Wenninger, G. N. McEwen, The Cosmetic, Toiletry, and Fragrance Association, 9^{th} edition, 2002) in Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive, and Skin Protectants, as well as all compounds listed in EP 0 934 956 (pp. 11-13) under water soluble conditioning agent and oil soluble conditioning agent. A portion of these compounds is listed under the constituents of the aqueous phase and the oil phase in particular. Further advantageous conditioners in accordance with the invention are, for example, compounds that are called polyquaternium in accordance with the International Nomenclature for Cosmetic Ingredients (INCI), in particular Polyquaternium-1 to Polyquaternium-56).

A particularly preferred embodiment of the present invention are textiles, which are impregnated with an emulsion of the invention. According to the invention, it is preferred that the textile be present in the form of a nonwoven fabric, in particular in the form of a wipe or “pad.”

According to the invention, a textile is preferred, which is produced from a blend of rayon fibers, polyester fibers, and/or cotton fibers.

It is especially advantageous for the purposes of the present invention to produce the textile fabric of the textile according to the invention from cotton fibers in a range from 1-30% by weight, rayon fibers in a range from 9-80% by weight, and polyester in a range from 19-90%, each based on the total weight of the textile.

In a very particularly preferred manner, the textile of the invention has on its surface a cotton content of up to 30% by weight and in its interior a cotton content of up to 10% by weight, each based on the total weight of the textile.

In accordance with the invention, however, it is also advantageous to use the emulsion as sprayable emulsion. In such a case, the emulsion of the invention can be applied with the aid of a pressure gas from an aerosol can or with the aid of a pump from a pump spray applicator (also known as pump atomizer).

In accordance with the invention, it is also advantageous to use a spray applicator in the form of a pump spray applicator or an aerosol can containing an emulsion according to one of the foregoing claims.

Advantageous in accordance with the invention is the use of the emulsion of the invention, or a textile, or a spray applicator for the care of the skin, in particular skin of the face, aged skin, dry or otherwise stressed skin, and in particular for the protection of the skin against UV radiation.

The following examples are intended to describe in greater detail the compositions according to the invention, without however intending to limit the invention to these examples. The numerical values in the examples denote weight percentages based on the total weight of the respective preparations.

### EXAMPLES

(1) Sprayable O/W Emulsion

| | % by weight |
|---|---|
| Ceteareth-20 | 2.00 |
| PEG-100 stearate | 1.00 |
| Glycerylstearate | 2.50 |
| Caprylic/capric triglyceride | 2.00 |
| Cetearyl alcohol | 1.50 |
| Octyl dodecanol | 1.00 |
| Dicaprylyl carbonate | 2.00 |
| Myristyl myristate | 1.00 |
| Dimethicone (linear) | 2.00 |
| Carageenan | 1.00 |
| Xanthan gum | 0.25 |
| Ethylhexyl triazone | 1.00 |
| Ethylhexylmethoxy cinnamate | 5.00 |
| Butylmethoxy dibenzoylmethane | 2.00 |
| EDTA | 0.20 |
| Tocopheryl acetate | 0.50 |
| Glycerin | 5.00 |
| Sodium chloride | 2.50 |
| Butylene glycol | 3.00 |
| Phenoxyethanol | 0.50 |
| Methylparaben | 0.30 |
| Propylparaben | 0.20 |
| Butylparaben | 0.05 |
| Fillers/additives (dye, BHT) | 0.20 |
| Perfume | q.s. |
| Water | 100.00 |
| PH = 6.0 | |
| Ethylhexyl triazone | |
| Diethylhexyl butamidotriazone | |

(2) Sprayable O/W Emulsion

| | % by weight |
|---|---|
| PEG-40 stearate | 2.50 |
| Glyceryl stearate | 1.50 |
| Cetearyl alcohol | 1.50 |
| Caprylic/capric triglyceride | 1.00 |
| Jojoba oil | 3.00 |
| Octyl dodecanol | 200 |
| Squalane | 2.00 |
| Dimethicone (linear) | 2.00 |
| Vaseline | 1.00 |
| Squalane | 1.00 |
| Octocrylene | 2.00 |
| Ethylhexylmethoxy cinnamate | 2.00 |
| Bis-ethylhexyloxyphenol- | 1.00 |
| methoxyphenyltriazine | |
| Xanthan gum | 0.10 |
| Carageenan | 1.00 |
| Panthenol | 1.00 |
| Tocopheryl acetate | 0.50 |
| Glycerin | 7.00 |
| Sodium chloride | 2.50 |
| Ethylhexyl glycerin | 0.50 |
| Phenoxyethanol | 0.50 |
| Methylparaben | 0.30 |
| Propylparaben | 0.20 |
| Butylparaben | 0.05 |
| Fillers/additives (dye, BHT, starch) | 0.20 |
| Perfume | q.s. |
| Water | 100.00 |
| PH = 6.0 | |

(3) Sprayable Sunscreen Emulsion

| | % by weight |
|---|---|
| PEG-40 stearate | 2.00 |
| Glyceryl stearate | 1.50 |
| Cetearyl alcohol | 1.00 |
| Caprylic/capric triglyceride | 2.00 |
| Shea butter | 1.00 |
| Octyl dodecanol | 2.00 |
| C12-15 alkylbenzoate | 2.00 |
| Dimethicone (linear) | 2.00 |
| Carageenan | 0.10 |
| Xanthan gum | 0.05 |
| PVP-hexadecene copolymer | 0.20 |
| Ethylhexylmethoxy cinnamate | 5.00 |
| Bis-Ethylhexyloxyphenol- | 2.00 |
| methoxyphenyltriazine | |
| Ethylhexyl triazone | 2.00 |
| Phenylbenzimidazole sulfonic acid | 2.00 |
| Creatine | 0.50 |
| Panthenol | 1.00 |
| Tocopheryl acetate | 0.50 |
| Glycerin | 5.00 |
| Methylpropanediol | 1.50 |
| Phenoxy ethanol | 0.50 |
| Methylparaben | 0.30 |
| Propylparaben | 0.10 |
| Ethylparaben | 0.10 |
| Butylparaben | 0.05 |
| Fillers/additives (dye, BHT) | 0.20 |
| Perfume | q.s. |
| Water | 100.00 |

PH = 7.0

(4) Sprayable Sunscreen Emulsion

| | % by weight |
|---|---|
| Ceteareth-20 | 2.00 |
| PEG-100 stearate | 1.00 |
| Glyceryl stearate | 2.50 |
| Caprylic/capric triglyceride | 2.00 |
| Cetearyl alcohol | 1.50 |
| C12-15 Alkylbenzoate | 2.00 |
| Dicaprylyl carbonate | 2.00 |
| Myristyl myristate | 1.00 |
| Dimethicone (linear) | 2.00 |
| Ethylhexyl triazone | 1.50 |
| Ethylhexylmethoxy cinnamate | 5.00 |
| Butylmethoxydibenzoyl methane | 2.00 |
| Diethylhexyl butamidotriazone | 1.00 |
| Phenylbenzimidazole sulfonic acid | 1.50 |
| Carageenan | 0.50 |
| Xanthan gum | 0.25 |
| PVP-hexadecene copolymer | 0.20 |
| EDTA | 0.20 |
| Tocopheryl acetate | 0.50 |
| Glycerin | 5.00 |
| Butylene glycol | 3.00 |
| Phenoxyethanol | 0.50 |
| Methylparaben | 0.30 |
| Propylparaben | 0.20 |
| Butylparaben | 0.05 |
| Fillers/additives(dye, BHT) | 0.20 |
| Perfume | q.s. |
| Water | 100.00 |

PH = 7.0

1. A cosmetic or dermatological oil-in-water (O/W) emulsion comprising:
a) one or more electrolytes in a concentration of 0.5 to 8% by weight,
b) a thickener in a concentration of 0.05 to 1% by weight, and
c) an emulsifier combination in a concentration of 0.5 to 6% by weight, said emulsifier combination comprising at least one polyethylene glycol stearate esters and at least one further emulsifier selected from the group consisting of polyoxyethylene cetylstearyl ethers and glyceryl stearate,
wherein the weight percentages are based on the total weight of the emulsion, and wherein said emulsion has a viscosity of 100 to 1900 mPa·s.

2. The emulsion as claimed in claim 1, wherein the emulsifier combination comprises PEG-100 stearate and ceteareth-20.

3. The emulsion as claimed in claim 1, wherein the emulsifier combination comprises PEG-40 stearate and glyceryl stearate.

4. The emulsion as claimed in claim 1, wherein the emulsifier combination comprises PEG-100 stearate, polyoxyethylene(20) cetylstearyl ether and glyceryl stearate.

5. The emulsion as claimed in claim 1, wherein said at least one further emulsifier includes at least one polyoxyethylene cetylstearyl ether, and wherein the weight ratio of at least one polyethylene glycol stearate esters to at least one polyoxyethylene cetylstearyl ethers is from 1:10 to 5:1.

6. The emulsion as claimed in claim 1, wherein said at least one further emulsifier includes glyceryl stearate, and wherein the weight ratio of the at least one polyethylene glycol stearate ester to glyceryl stearate is from 1:10 to 3:1.

7. The emulsion as claimed in claim 1, wherein the thickener comprises at least one compound selected from the group consisting of carrageenan, xanthan gum, polyacrylate, cellulose ether, acryloyl dimethyl taurate/VP copolymer, and hydroxypropyl distarch phosphate.

8. The emulsion as claimed in claim 1, wherein the one or more electrolytes includes at least one electrolyte selected from the group consisting of sodium chloride, potassium chloride, magnesium chloride, taurine, citric acid, lactic acid, ascorbic acid, ascorbic acid phosphate ester, creatine, salicylic acid, and carnitine.

9. The emulsion as claimed in claim 1, further comprising at least one UV light protective filter substance.

10. A cosmetic or dermatological product comprising:
a spray applicator in the form of a pump spray applicator or an aerosol can; and
a cosmetic or dermatological oil-in-water emulsion provided within the spray applicator comprising:
a) one or more electrolytes in a concentration of 0.5 to 8% by weight,
b) a thickener in a concentration of 0.05 to 1% by weight, and
c) an emulsifier combination in a concentration of 0.5 to 6% by weight, said emulsifier combination comprising at least one polyethylene glycol stearate ester and at least one further emulsifier selected from the group consisting of polyoxyethylene cetylstearyl ethers and glyceryl stearate,
wherein the weight percentages are based on the total weight of the emulsion,
wherein said emulsion has a viscosity of 100 to 1900 mPa·s, and
wherein said emulsion is capable of being sprayed.

11. The emulsion as claimed in claim 8, wherein said at least one UV filter substance includes phenylene-1,4-bis-(2-benzimidazyl)-3,3′-5,5′tetrasulfonic acid-bis-sodium salt.

12. The emulsion as claimed in claim 1, further comprising at least one cosmetic active ingredient.

13. The emulsion as claimed in claim 1, wherein said thickener includes at least one homopolymer or copolymer of acrylic acid with the structure 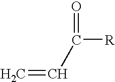
wherein R is —OH, —OR′, or —NHR′, and wherein R′ is selected from the group consisting of alkyl-, aryl-, heteroalkyl-, heteroaryl-, and alkyl sulfonates.

14. The emulsion as claimed in claim 1, wherein said thickener includes hydroxypropylmethyl cellulose in a concentration of from 0.5 to 2% by weight.

15. The emulsion as claimed in claim 1, wherein said emulsifier combination includes glyceryl stearate citrate in a concentration from 0.5 to 3% by weight, and wherein said thickener includes a combination of carrageenan and xanthan gum, wherein the ratio of carrageenan to xanthan gum is from 1:5 to 5:1.

16. The emulsion as claimed in claim 1, further comprising at least one film former.

17. The emulsion as claimed in claim 16, wherein the at least one film former is selected from the group consisting of polyvinyl pyrrolidone hexadecane copolymer and polyvinyl pyrrolidone eicosene copolymer.

18. The emulsion as claimed in claim 8, wherein the one or more electrolytes includes phenyl benzimidazole sulfonic acid.

19. The emulsion as claimed in claim 9, wherein said at least one UV light protective filter substance includes at least one UV filter substance selected from the group consisting of dibenzoylmethane derivatives; benzotriazole derivatives; phenylene-1,4-bis(2-benzimidazyl)-1,3′-5,5′-tetrasulfonic acid; salts of phenylene-1,4-bis(2-benzimidazyl)-1,3′-5,5′-tetrasulfonic acid; 1,4-d(2-oxo-10-sulfo-3-bornylidenemethyl)-benzene; salts of 1,4-d(2-oxo-10-sulfo-3-bornylidenemethyl)-benzene; 2,4-bis {[4-(2-ethyl-hexyloxy)-2-hydroxy]phenyl}-6-(4-methoxyphenyl)-1,3,5-triazine; and dioctylbutylamidotriazone.

20. A cosmetic or dermatological product comprising:
a textile, and
a cosmetic or dermatological oil-in-water emulsion comprising:
a) one or more electrolytes in a concentration of 0.5 to 8% by weight,
b) a thickener in a concentration of 0.05 to 1% by weight, and
c) an emulsifier combination in a concentration of 0.5 to 6% by weight, said emulsifier combination comprising at least one polyethylene glycol stearate esters and at least one further emulsifier selected from the group consisting of polyoxyethylene cetylstearyl ethers and glyceryl stearate,
wherein the weight percentages are based on the total weight of the emulsion, and
wherein said emulsion has a viscosity of 100 to 1900 mPa·s, and
wherein said textile is impregnated with said emulsion.
